(19) United States
(12) Patent Application Publication
(10) Pub. No.: US 2023/0284951 A1
(43) Pub. Date: Sep. 14, 2023

(54) BIOMAGNETIC FIELD MEASUREMENT SYSTEM, METHOD FOR CONTROLLING BIOMAGNETIC FIELD MEASUREMENT SYSTEM, AND RECORDING MEDIUM STORING CONTROL PROGRAM FOR BIOMAGNETIC FIELD MEASUREMENT SYSTEM

(71) Applicants: Koki Ishida, Kanagawa (JP); Taishi Watanabe, Tokyo (JP); Shigenori Kawabata, Tokyo (JP); Yuta Tanaka, Tokyo (JP)

(72) Inventors: Koki Ishida, Kanagawa (JP); Taishi Watanabe, Tokyo (JP); Shigenori Kawabata, Tokyo (JP); Yuta Tanaka, Tokyo (JP)

(73) Assignees: Ricoh Company, Ltd., Tokyo (JP); National University Corporation Tokyo Medical and Dental University, Tokyo (JP)

(21) Appl. No.: 18/178,759

(22) Filed: Mar. 6, 2023

(30) Foreign Application Priority Data
Mar. 10, 2022 (JP) 2022-037446
Apr. 12, 2022 (JP) 2022-065586

(51) Int. Cl. A61B 5/243; A61B 5/00

(52) U.S. Cl. CPC A61B 5/243; A61B 5/743

(57) ABSTRACT

The present invention is designed such that, even when inward currents cannot be calculated properly, it is possible to generate current waveforms that can appropriately evaluate the action of a target body tissue by calculating currents that are equivalent to inward currents. A biomagnetic field measurement system includes circuitry and a memory storing executable instructions which, when executed by the circuitry, cause the circuitry to: based on current components extracted from current signals calculated from a biomagnetic field signal, add up current waveforms of current components along a conduction pathway of an action current in a body tissue that is targeted for evaluation, and generate a current waveform for evaluating an intra-cellular current flowing in the conduction pathway, locations of the current components of the added current waveforms being predetermined set distances apart on the conduction pathway, in front of and behind a location of interest on the conduction pathway.

### CROSS-REFERENCE TO RELATED APPLICATIONS

The present application claims priority under 35 U.S.C. § 119 to Japanese Patent Application No. 2022-037446, filed Mar. 10, 2022, and Japanese Patent Application No. 2022-065586, filed Apr. 12, 2022, the contents of which are incorporated herein by reference in their entirety.

### BACKGROUND OF THE INVENTION

### 1. Field of the Invention

The present disclosure relates to a biomagnetic field measurement system, a method for controlling the biomagnetic field measurement system, and a recording medium storing control programs for the biomagnetic field measurement system.

### 2. Description of the Related Art

For example, a biomagnetic field measurement system estimates the distribution of currents inside the subject's body. The biomagnetic field measurement system estimates this by using spatial filtering or the like, based on magnetic field data obtained by measuring biomagnetic fields with a magnetic field measurement device. Then, for example, in a morphological image of a portion of the subject's body tissue that is targeted for measurement, the biomagnetic field measurement system generates current waveforms on multiple virtual electrodes that are arranged to follow the conduction of action currents in the target body tissue, based on the estimated distribution of currents, and displays the generated current waveforms on a display device. This allows visualization of electrical action at any location in the body. The body tissue here refers to, for example, the nerve axon, skeletal muscle, cardiac muscle, or smooth muscle.

### RELATED-ART DOCUMENT

[Patent Document]
[Patent Document 1] Unexamined Japanese Patent Application Publication No. 2021-83479

### SUMMARY OF THE INVENTION

Action currents derived from depolarization can be roughly divided into intra-cellular currents that flow in a given body tissue, and volume currents that flow outside the given body tissue. Among the volume currents, especially the currents that flow toward the depolarization site will be hereinafter referred to as “inward currents.” Note that, in an action of a target body tissue, currents flow in opposite directions from the depolarization part along the inside of cells of a specific tissue, so that the depolarization site travels. The direction in which the depolarization site travels will be hereinafter referred to as the “front side,” and terms such as “front,” “forward,” “in front of” and so forth will be used to refer to this direction. On the other hand, terms such as “the back side,” “back,” “backward,” “rearward,” “behind” and so forth will be used to refer to the opposite of the above travelling direction of the depolarization site. For example, in a joint, the inward currents may not be calculated properly due to the influence of non-conductors such as bones. In this case, the shape of inward current waveforms that are derived from extracted inward currents is deformed. Normally, when a body tissue's function is evaluated in detail, inward current waveforms are used. Therefore, if inward currents cannot be calculated properly, it then becomes difficult to evaluate the function of the target body tissue appropriately.

The present disclosure has been made in view of the above, and aims to generate current waveforms that can be used to evaluate the action of a target body tissue appropriately, by calculating currents that are equivalent to inward currents even when inward currents cannot be calculated properly.

In order to achieve the above aim, according to one embodiment of the present disclosure, a biomagnetic field measurement system has: circuitry; and a memory storing executable instructions which, when executed by the circuitry, cause the circuitry to: based on current components extracted from current signals calculated from a biomagnetic field signal, add up current waveforms of current components along a conduction pathway of an action current in a body tissue that is targeted for evaluation, and generate a current waveform for evaluating an intra-cellular current flowing in the conduction pathway, locations of the current components of the added current waveforms being predetermined set distances apart on the conduction pathway, in front of and behind a location of interest on the conduction pathway.

Thus, currents that are equivalent to inward currents are calculated even when inward currents cannot be calculated properly, so that it is possible to generate current waveforms that can appropriately evaluate the action of a target body tissue.

### BRIEF DESCRIPTION OF THE DRAWINGS

FIG. 1 is a block diagram that illustrates an example of a biomagnetic field measurement system including a biomagnetic field measurement system according to an embodiment of the present disclosure;

FIG. 2 is a diagram that explains an example model of action currents in a body tissue;

FIG. 3 is a diagram that explains an example method for calculating current intensity at virtual electrodes generated by the virtual electrode generating part of FIG. 1;

FIG. 4 is a diagram that explains examples of various current waveforms in the ulnar nerve of a left arm when an electrical stimulation is applied to the left wrist of a subject (healthy person);

FIG. 5 is a diagram that explains an example of generating a summation waveform of intra-axonal currents from current waveforms of an intra-axonal current calculated at a back location and a front location with respect to each first virtual electrode;

FIG. 6 is an enlarged view that illustrates the current waveforms of inward currents and summation intra-axonal currents shown in FIG. 4;

FIG. 7 is a diagram that explains examples of various current waveforms in the ulnar nerve of a right arm when an electrical stimulation is applied to the right wrist of the subject (patient);

FIG. 8 is an enlarged view that illustrates current waveforms of the inward current and the summation intra-axonal current shown in FIG. 7;

FIG. 9 is a diagram that illustrates examples of waveforms of inward currents and summation waveforms of intra-axonal currents at the left elbow when a stimulation is applied to the ulnar nerve at the left hand of a healthy person;

FIG. 10 is a diagram that illustrates the results of a clinical experiment with 5 healthy people;

FIG. 11 is a flowchart that illustrates an example of the operation of the arithmetic part of FIG. 1;

FIG. 12 is a flowchart that illustrates the continuation of the operation of FIG. 11;

FIG. 13 is a flowchart that illustrates the continuation of the operation of FIG. 12; and

FIG. 14 is a block diagram that illustrates an example hardware structure of the data processing device of FIG. 1.

### DESCRIPTION OF EMBODIMENTS

Hereinafter, an embodiment of the present disclosure will be described with reference to the accompanying drawings. Note that, throughout the accompanying drawings, the same components will be assigned the same reference codes, and redundant description will be omitted.

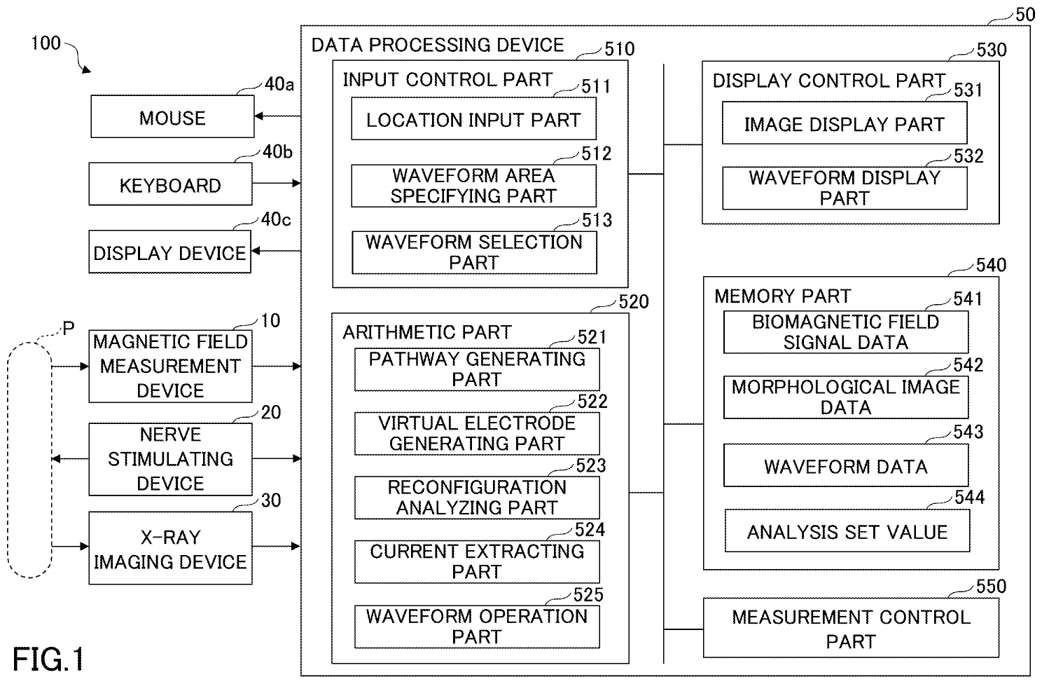

FIG. 1 is a block diagram that illustrates an example of a biomagnetic field measurement system including a biomagnetic field measurement system according to a first embodiment of the present disclosure. The biomagnetic field measurement system 100 shown in FIG. 1 includes a magnetic field measurement device 10, a nerve stimulating device 20, an X-ray imaging device 30, a mouse 40a, a keyboard 40b, a display device 40c, and a data processing device 50 that functions as a biomagnetic field measurement system.

For example, the magnetic field measurement device 10 has a SQUID sensor array and a signal processing device. The SQUID sensor array includes multiple superconducting quantum interference devices (SQUIDs). The magnetic field measurement device 10 can measure the biomagnetic field that is induced in an evaluation-target body tissue of a subject P by an electrical stimulation from the nerve stimulating device 20.

For example, the magnetic field measurement device 10 is used as a magnetospinograph (MSG) or a magnetomyograph (MMG). Note that the magnetic field measurement device 10 can be used as a magnetoencephalograph (MEG) or a magnetocardiograph (MCG). In the following description, a superconducting quantum interference device may be also referred to as a “SQUID.”

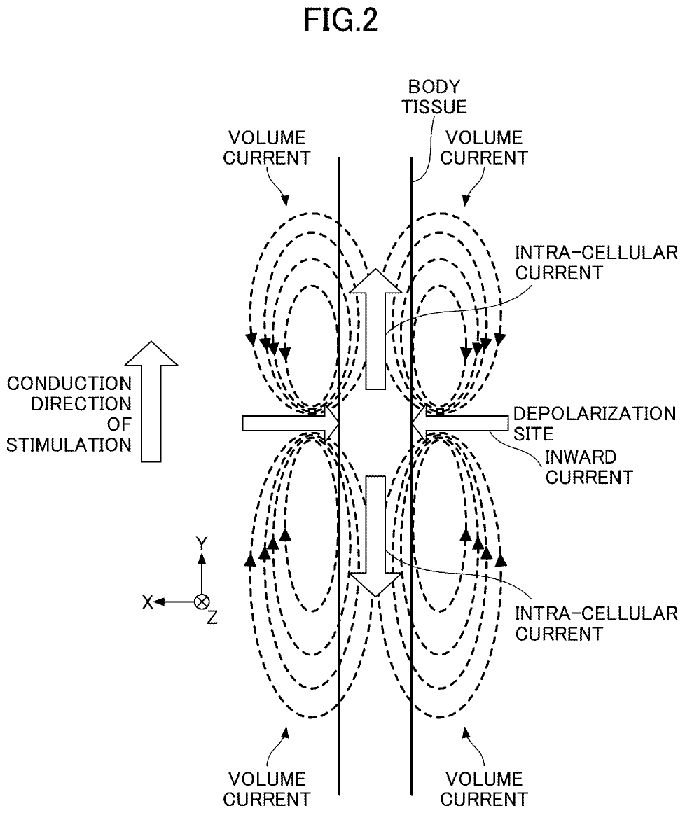

The nerve stimulating device 20 electrically stimulates the body tissues of the subject P through electrodes placed on the body surface (skin) of the subject P. The X-ray imaging device 30 takes a morphological image of a biomagnetic field measurement target portion of the subject P. Here, the measurement target portion refers to a portion where currents flow in opposing directions from the depolarization site shown in FIG. 2, along the inside of cells of a specific tissue, and where the depolarization site travels. The specific tissue is, for example, the nerve axon, skeletal muscle, cardiac muscle, or smooth muscle. Note that, when measuring a biomagnetic field induced in a muscle of the subject P, an MRI (Magnetic Resonance Imaging) device or a CT (Computed Tomography) device may be used, instead of the X-ray imaging device 30. Alternatively, a morphological image of the subject P may be taken by using a combination of the X-ray imaging device 30 with an MRI device and/or a CT device (not shown).

The data processing device 50 has a function of controlling the timing of applying an electrical stimulation to the living body by the nerve stimulating device 20 and a function of executing information processing on biological information such as the biomagnetic field measured by the magnetic field measurement device 10. Also, the data processing device 50 has a function of controlling the X-ray imaging of the subject P by the X-ray imaging device 30. The data processing device 50 has a function of receiving inputs from the input/output devices such as the mouse 40a and the keyboard 40b.

Furthermore, the data processing device 50 has a function of superimposing the directions of currents generated according to the magnetic field measured by the magnetic field measurement device 10, over an X-ray image, and displaying the resulting image on the display device 40c. With this image displayed on the display device 40c, the data processing device 50 has a function of calculating changes in current values, over time, at multiple consecutive locations (virtual electrodes) specified by the user by operating the mouse 40a.

For example, the user uses the mouse 40a to enter multiple locations along the conduction pathway of an action current in a body tissue, identified in the X-ray image displayed on the screen of the display device 40c. The data processing device 50 has, for example, a function of generating a curve such as a Bezier curve along the body tissue based on the multiple locations entered by the user, and generating multiple virtual electrodes along the curve based on set values that are provided in advance. Furthermore, the data processing device 50 has a function of displaying the current waveforms at the virtual electrodes, per virtual electrode, on the display device 40c.

The data processing device 50 has an input control part 510, an arithmetic part 520, a display control part 530, a memory part 540, and a measurement control part 550. For example, the functions of the input control part 510, the arithmetic part 520, and the display control part 530 are implemented by control programs executed on a processor such as a CPU (Central Processing Unit) installed in the data processing device 50.

Note that the input control part 510, the arithmetic part 520, the display control part 530, and the measurement control part 550 may be implemented by hardware such as an FPGA, or may be implemented by a combination of software and hardware.

For example, the memory part 540 is implemented by at least one semiconductor memory device such as a DRAM (Dynamic Random Access Memory), an SRAM (Static Random Access Memory), a ROM (Read Only Memory), and a flash memory. Note that the memory part 540 may be implemented by including a semiconductor memory device and an HDD (Hard Disk Drive) or an SSD (Solid State Drive).

The input control part 510 has a location input part 511, a waveform area specifying part 512, and a waveform selection part 513. The arithmetic part 520 has a pathway generating part 521, a virtual electrode generating part 522, a reconfiguration analyzing part 523, a current extracting part 524, and a waveform operation part 525. The display control part 530 has an image display part 531 and a waveform display part 532. The memory part 540 is assigned a memory field for storing biomagnetic field signal data 541, morphological image data 542, waveform data 543, analysis set values 544, and so forth.

The input control part 510 receives as input the operations of the mouse 40a, the keyboard 40b, and so forth, by the user of the magnetic field measurement device 10. Inputting information through operations of the mouse 40a and the keyboard 40b is an example of “external input.” Also, the input control part 510 receives as input the biomagnetic field signals measured by the magnetic field measurement device 10 as data, and stores the input data in the memory part 540. The input control part 510 receives as input the X-ray image data captured by the X-ray imaging device 30, and stores the input data in the memory part 540.

The location input part 511 receives as input the location of the body tissue for evaluating the biomagnetic field on the morphological image, such as an X-ray image, that is displayed on the screen of the display device 40c. The location information received as input by the location input part 511 is stored in the memory part 540 as analysis set values 544.

The location input part 511 receives as input the distances (first distance and second distance) from one of multiple virtual electrodes set on the path of the action current in the body tissue, to both predetermined locations behind and in front of, or on the back side and the front side of, the virtual electrode on the path of the action current in the body tissue. The back side on the path of a body tissue's action current is an example of one of the front side and the back side, and the front side on the path of a body tissue's action current is an example of the other side of the front side and the back side. The first distance and the second distance are examples of set distances that are configured in advance, and are input by using, for example, the numeric keypad of the keyboard. Note that the evaluator such as a doctor may enter the distances into AI (Artificial Intelligence) that is trained to calculate appropriate first distances and second distances, and the first distance and the second distance may be determined by AI. In this case, the location input part 511 may automatically receive as input the first distance and the second distance determined by AI. Furthermore, the first distance and the second distance determined by AI may be presented to the evaluator such as a doctor by displaying them on the display device 40c as reference values for assisting diagnosis.

The waveform area specifying part 512 receives as input the range of time to display the current waveforms on the virtual electrodes on the display device 40c. The waveform selection part 513 can select the current waveforms to display on the display device 40c through operations of, for example, the mouse 40a and the keyboard 40b. Furthermore, the waveform selection part 513 can select the current waveforms to superimpose and display, amongst the current waveforms displayed on the display device 40c, through operations of, for example, the mouse 40a and the keyboard 40b.

The pathway generating part 521 calculates the path of the body tissue based on location information, which is input from the location input part 511 and which indicates the location of the body tissue in the morphological image. Below, the path of the action current in the body tissue calculated by the pathway generating part 521 will be also referred to as the “conduction pathway of the action current.” Here, the conduction pathway of the action current calculated by the pathway generating part 521 is represented by, for example, multiple sets of coordinate information or an equation that represents a curve, and is stored in the memory part 540 as analysis set values 544.

The virtual electrode generating part 522 generates multiple first virtual electrodes, at regular intervals, for example, on the conduction pathway of the action current calculated by the pathway generating part 521. The locations of the first virtual electrodes are an example of locations of interest on the conduction pathway of the action current. Note that, depending on user settings, the first virtual electrodes can be set at any locations on the body tissue pathway. In this case, the locations of interest on the conduction pathway of the action current can be set at any locations on the conduction pathway of the action current. Furthermore, at each first virtual electrode, the virtual electrode generating part 522 generates second virtual electrodes, predetermined distances apart from the first virtual electrode, on both sides in the direction orthogonal to the conduction direction of the action current in the body tissue.

The number and spacing of the first virtual electrodes generated on the conduction pathway of the action current and the distance between each first virtual electrode and second virtual electrode are entered in advance by the user via the mouse 40a, the keyboard 40b, or the like. Then, the location information of the first virtual electrodes and the second virtual electrodes are stored in the memory part 540, as analysis set value 544, by the input control part 510. Hereinafter, when the first virtual electrodes and the second virtual electrodes are described without distinction, they will be simply referred to as “virtual electrodes.”

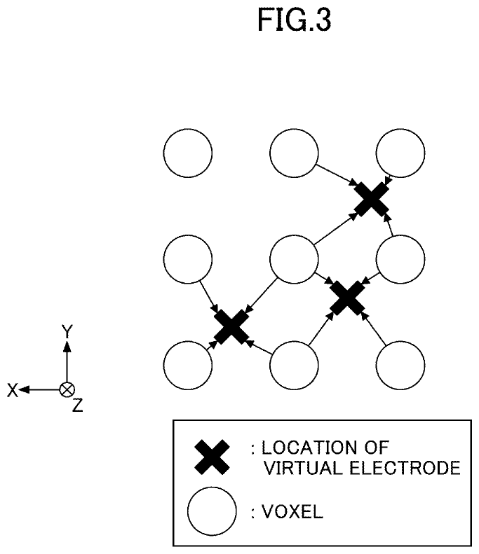

The reconfiguration analyzing part 523, using the subject P's biomagnetic field data (biomagnetic field signals) obtained by measuring the biomagnetic field with the magnetic field measurement device 10, reconfigures the current components (current signals) on a per voxel basis, where the voxels are arranged in a matrix at predetermined intervals. That is, the reconfiguration analyzing part 523 calculates current signals based on biomagnetic field signals. The voxels will be described with reference to FIG. 3.

Based on the location relationship between each virtual electrode and voxel, the current extracting part 524 extracts the current component of each virtual electrode by using the current components at the voxels calculated by the reconfiguration analyzing part 523. For example, in the time range received as input at the waveform area specifying part 512, the current extracting part 524 extracts the current components (where, for example, the front side is positive and the back side is negative) at the first virtual electrodes along the nerve pathway as intra-cellular currents that travel in the body tissue along the nerve pathway. Also, in the time range received as input at the waveform area specifying part 512, the current extracting part 524 extracts, as inward currents, the current components that are headed for the body tissue from the second virtual electrodes located around the body tissue.

Furthermore, the current extracting part 524 extracts, as intra-cellular currents, the current components at locations that are the first distance and the second distance forward and backward apart from each first virtual electrode on the body tissue. Below, the location that is the first distance backward apart from each first virtual electrode on the nerve will be referred to as the “back location,” and the location that is the second distance forward apart from each first virtual electrode on the nerve will be referred to as the “front location.”

The waveform operation part 525 generates current waveforms that show changes, over time, with the intra-cellular currents at each first virtual electrode, extracted by the current extracting part 524. Also, the waveform operation part 525 generates current waveforms that show the changes, over time, with the inward currents at each second virtual electrode, extracted by the current extracting part 524. Of the volume currents flowing outside the body tissue, inward currents, which are current components that flow into the depolarization site, are important in the evaluation of nerve function.

Furthermore, the waveform operation part 525 generates current waveforms that show the changes of intra-cellular currents over time at the back location and the front location. The waveform operation part 525 inverts the sign of the current waveform of the back location, adds this inverted current waveform to the current waveform of the front location, and thus generates a summation waveform of intra-cellular currents.

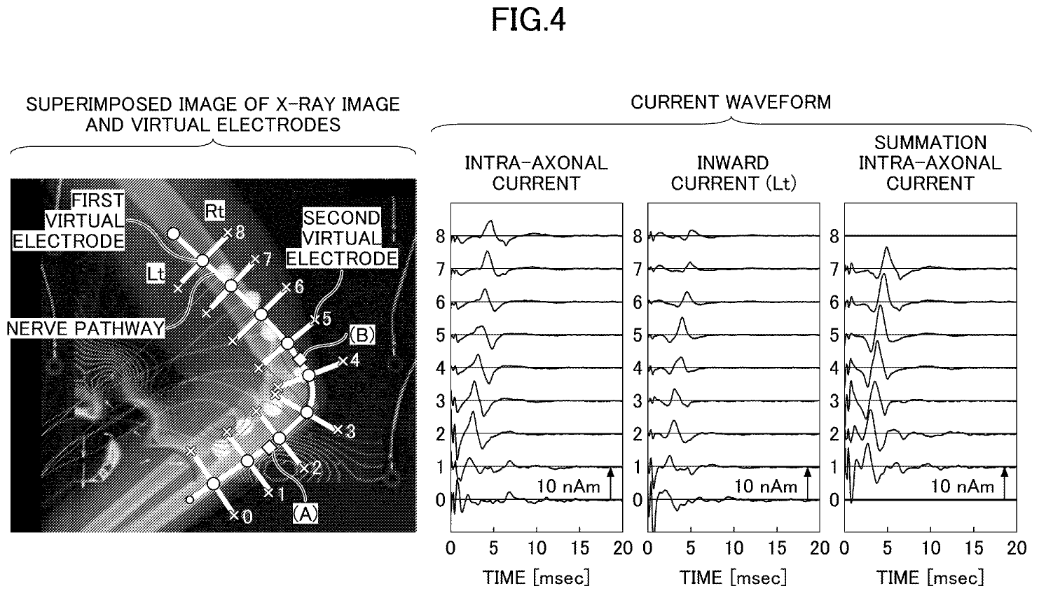

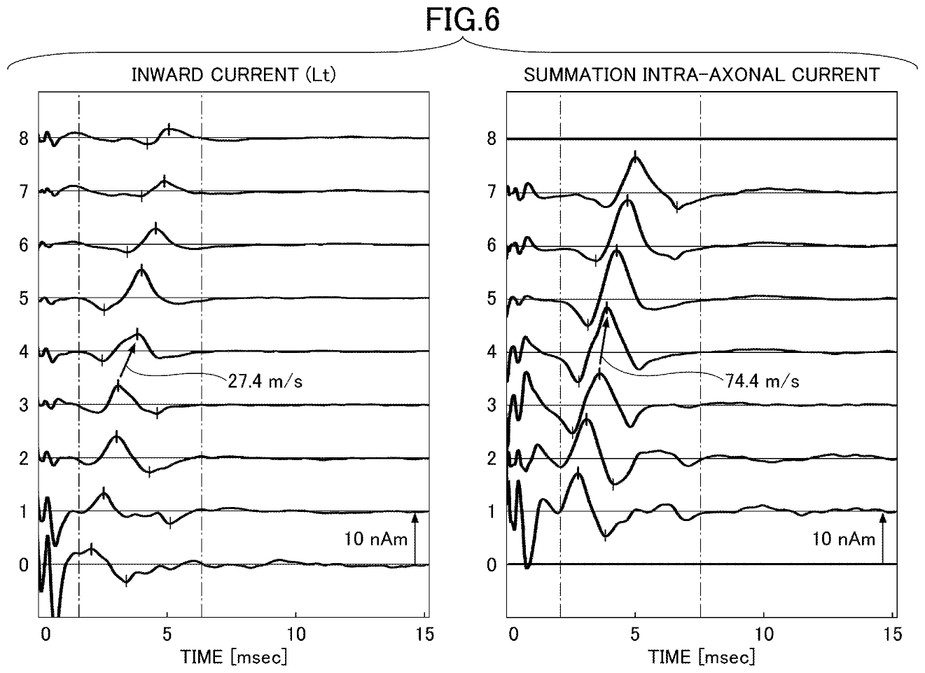

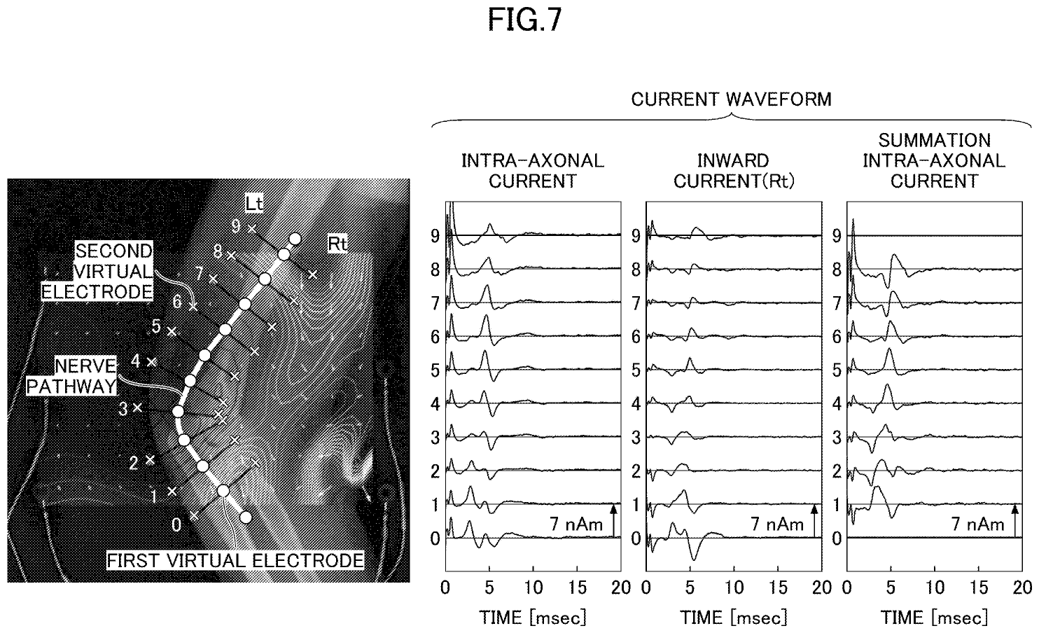

As shown in FIG. 4 and FIG. 7 to be described later, the image display part 531 superimposes small white arrows that indicate the directions and intensity of currents at each voxel, reconfigured by the reconfiguration analyzing part 523, on the morphological image (X-ray image), and displays the resulting image on the display device 40c. Also, as shown in FIG. 4 and FIG. 6, the image display part 531 superimposes the nerve route calculated by the pathway generating part 521 and the virtual electrodes generated by the virtual electrode generating part 522 over the X-ray image, and displays the resulting image on the display device 40c.

The waveform display part 532 arranges the current data values of the intra-cellular currents and the current data values of the inward currents in chronological order, so as to display image data of current waveforms that show the changes of the intra-cellular currents and the inward currents at the virtual electrodes over time. Then, the waveform display part 532 associates the current waveforms, generated on a per virtual electrode basis, with the virtual electrodes superimposed on the X-ray image, and displays these on the display device 40c.

Furthermore, the waveform display part 532 is capable of displaying a summation waveform of intra-cellular currents, in addition to the X-ray images including the superimposed image, the intra-cellular current waveforms, and the inward current waveforms. Note that the waveform display part 532 displays any images or current waveforms among the X-ray image including the superimposed image, the intra-cellular current waveforms, the inward current waveforms and the summation waveform of intra-cellular currents based on what is received as input at the input control part 510.

The measurement control part 550 controls the magnetic field measurement by the magnetic field measurement device 10. Note that the measurement control part 550 may control the operation of the nerve stimulating device 20 or control the X-ray imaging by the X-ray imaging device 30.

The memory field of the biomagnetic field signal data 541 stores the magnetic field data, obtained by measuring the magnetic field derived from the subject P with the magnetic field measurement device 10. The memory field of the morphological image data 542 stores the X-ray image data of the magnetic field measurement-target part of the subject P, captured by the X-ray imaging device 30. The memory field of the waveform data 543 stores the waveform data generated by the waveform operation part 525.

In the memory field of the analysis set value 544, various parameters necessary for the measurement of the biomagnetic field by the magnetic field measurement device 10, and various set values such as ones for the filters (high-pass filter, low-pass filter, etc.) for use for the magnetic field data obtained by measuring the biomagnetic field, are stored in advance. Also, in the memory field of the analysis set value 544, location information to indicate the locations of voxels, which serve as current-calculating points on the image displayed on the display device 40c, and the locations of virtual electrodes for acquiring current waveforms, is stored in advance. Furthermore, in the memory field of the analysis set value 544, distance information to indicate the first distance and the second distance, which are distances from each first virtual electrode to the back location and the front location on the body tissue, is stored.

FIG. 2 is a diagram that explains an example of an action current model in a body tissue. FIG. 2 shows how currents are generated from the action of a body tissue that runs vertically straight in the drawing. The front side in the direction in which a stimulation travels (upward arrow) in FIG. 2 is the direction in which the depolarization site travels, and the back side in FIG. 2 is its opposite. For example, when an electrical stimulation is applied to a nerve, the stimulation produces electric currents that travel through the body tissue from back to front.

At this time, the intra-cellular current that flows forward in FIG. 2, the intra-cellular current that flows rearward in FIG. 2, and volume currents, which are electrical components that flow outside the body tissue and return to the depolarization site, are produced. The intra-cellular current that flows forward in FIG. 2 is referred to as the “leading component,” and the intra-cellular current that flows rearward in FIG. 2 is referred to as the “trailing component.”

In order to evaluate nerve function in detail, for example, the current extracting part 524 extracts the current components of intra-cellular currents that flow along the body tissue, and the current components of inward currents that flow into the depolarization site, based on current components that are reconfigured on a per voxel basis. The intra-cellular currents are current components in the direction that runs along the nerve pathway, and the inward currents are current components that travel toward the body tissue from outside the body tissue. For example, the inward currents are current components that travel toward the body tissue following the transverse direction of the body tissue.

Here, the law of conservation of electric charge applies to the action currents in body tissues. Therefore, the total amount of inward currents that flow into a body tissue is equal to the total amount of intra-cellular currents that flow in the body tissue. It then follows that the behavior of inward currents that travel toward a depolarization site can be represented by adding the intra-cellular currents at a location a predetermined distance backward apart from the depolarization site and the intra-cellular currents at a location a predetermined distance forward apart from the depolarization site.

FIG. 3 is a diagram that explains an example method of calculating the intensity of currents at virtual electrodes generated by the virtual electrode generating part 522 of FIG. 1. Referring to FIG. 3, by using the linear interpolation technique, the intensity of currents at virtual electrodes is calculated from the intensity of currents reconfigured on voxels arranged in a matrix. By this operation, the estimated intensity of currents in the X, Y, and Z directions of the virtual electrodes can be calculated.

Note that, in FIG. 3, the interval between voxels and the interval between virtual electrodes are set approximately the same for ease of explanation. In reality, however, the interval between virtual electrodes may be several times the interval between voxels and can be set in various ways. Also, for example, SQUID magnetic sensors are arranged at intervals of several centimeters, whereas voxels are arranged at intervals of several millimeters.

Note that the current intensity at virtual electrodes may be calculated by using the RENS (REcursive Null Steering) filtering technique developed by the present inventors. In this case, the current intensity at each virtual electrode can be calculated accurately, in a short time, compared to the case in which linear interpolation is used.

FIG. 4 is a diagram that explains examples of various current waveforms in the ulnar nerve of a left arm when an electrical stimulation is applied to the left wrist of a subject (healthy person). FIG. 4 shows an example of an image that is displayed on the display device 40c of FIG. 1. The left side of FIG. 4 shows a superimposed image, in which a nerve pathway, first virtual electrodes, and second virtual electrodes are superimposed on an X-ray image of the subject P's left arm. The nerve pathway is an example of a conduction pathway of action currents in body tissues. The superimposed image also contains small white arrows, which represent the directions and intensity of currents at voxels arranged in a matrix. Furthermore, the superimposed image contains current intensity distribution lines (contour-like curves), which indicate locations of the same current intensity. As for the current intensity distribution lines, a whiter color indicates a stronger current, and a darker color indicates a weaker current.

In FIG. 4, the first virtual electrodes are indicated by circles, and the second virtual electrodes are indicated by the symbol “X.” The second virtual electrodes are placed in the transverse direction of the nerve axon (for each first virtual electrode). This makes it possible to generate current waveforms of inward currents in the transverse direction of the nerve axon based on current components of the second virtual electrodes.

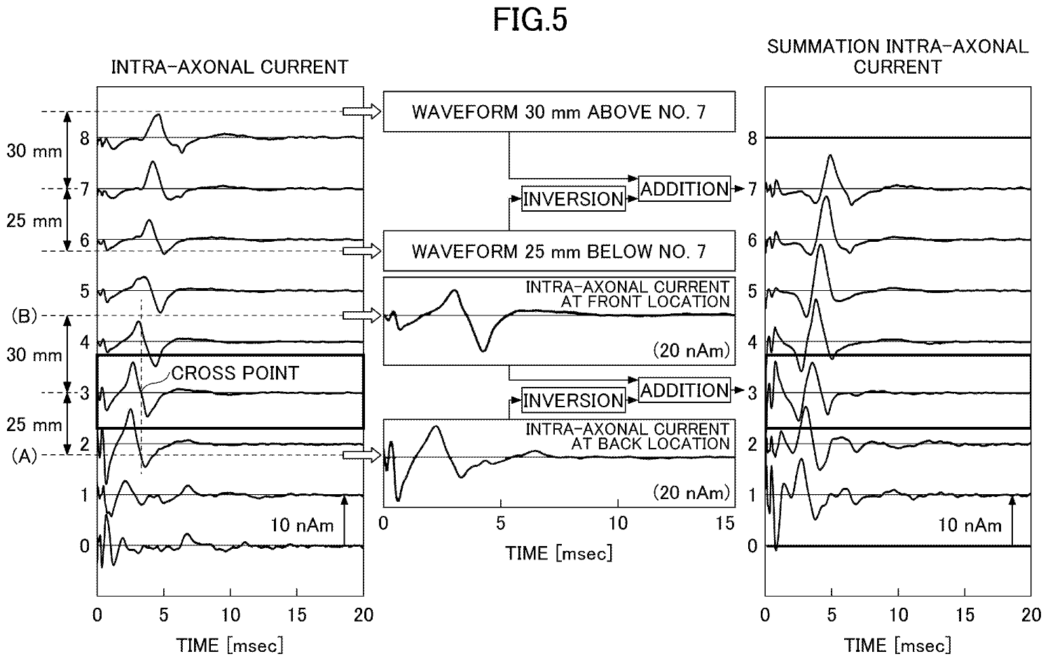

The reference code “Lt” indicates the line of second virtual electrodes located on the left side in the superimposed image displayed on the display device 40c. The reference code “Rt” indicates the line of second virtual electrodes located on the right side in the superimposed image displayed on the display device 40c. The numerical values 0 to 8 assigned to the virtual electrodes in the line Rt are electrode numbers that indicate the correspondence of the virtual electrodes with their respective current waveforms. The electrode numbers of the second virtual electrodes and the electrode numbers of the first virtual electrodes in the line Lt are the same as the corresponding electrode numbers of the second virtual electrodes in the line Rt. The reference codes “(A)” and “(B)” shown in the superimposed image indicate, respectively, the back location and the front location on the nerve axon, indicated by the reference codes (A) and (B) in FIG. 5.

The terminals connected to cables shown on the left and right sides in the X-ray image are electrodes provided so as to associate the X-ray image (morphological image) with the measurement locations of magnetic field data by the SQUID magnetic sensor, and photographed together with the subject P. The white circles (the white circles that are larger than the circles representing the first virtual electrodes) arranged at intervals along the ulnar nerve in the X-ray image indicate electrodes for potential measurement, placed for reference.

The right side of FIG. 4 shows the current waveforms of intra-axonal currents at respective first virtual electrodes, the current waveforms at respective second virtual electrodes indicated by the reference code Lt, and the current waveforms of summation intra-axonal currents, in which the intra-axonal currents at the back location (A) and the front location (B) are added up. The intra-axonal currents are an example of an intra-cellular current when a nerve is the target body tissue, and the summation intra-axonal current is an example of a summation intra-cellular current. The current waveform of the summation intra-axonal current is an example of a current waveform for evaluating an intra-cellular current. The intra-axonal current shown next to the superimposed image includes both current components of the intra-axonal current that flows forward shown in FIG. 2 and the intra-axonal current that flows rearward in FIG. 2.

The current waveform of each second virtual electrode shows changes of inward currents that travel through the nerve axon over time. The method of generating the current waveforms of the summation intra-axonal current will be described later with reference to FIG. 5. The numerical value (“10 nAm” in this example) shown in a lower right part of the three graphs of current waveforms indicates the current dipole (intensity).

FIG. 5 is a diagram that explains an example of generating a current waveform of a summation intra-axonal current from current waveforms of intra-axonal currents calculated at the back location and the front location with respect to each first virtual electrode. In the example shown in FIG. 5, the back location (for example, (A)) is set at a location 25 mm apart from each first virtual electrode, behind the ulnar nerve. The front location (for example, (B)) is set at a location 30 mm apart from each first virtual electrode, in front of the ulnar nerve. The back location and the front location are set by the evaluator such as a doctor who evaluates nerve function by observing the current waveforms of intra-axonal currents displayed on the display device 40c.

For example, the evaluator estimates, from the intra-axonal current waveform of interest of a first virtual electrode of interest, which is one of the first virtual electrodes set on the ulnar nerve in the morphological image, other intra-axonal current waveforms that peak at the crossing point where the current value of the intra-axonal current waveform becomes zero. These other intra-axonal current waveforms are current waveforms of intra-axonal currents calculated at the back location and the front location of the first virtual electrode of interest.

Then, using the keyboard 40b or the like, the evaluator enters the distances from the first virtual electrode of interest to the back location and the front location where the estimated intra-axonal current waveforms are obtained (in this example, the first distance=25 mm and the second distance=30 mm). This allows the evaluator to set a back location and a front location where appropriate summation waveforms of intra-axonal currents are obtained, based on what he/she sees and learns. Note that the distances to the back location and the front location may be set per first virtual electrode.

The current extracting part 524 extracts the current component of the trailing intra-axonal current at the back location that is the first distance apart from each first virtual electrode, and extracts the leading intra-axonal current component at the front location that is the second distance apart from each first virtual electrode. The waveform operation part 525 generates the current waveform at the back location based on the summation intra-axonal current extracted by the current extracting part 524 at the back location. Also, the waveform operation part 525 generates the current waveform at the front location based on the summation intra-axonal current at the front location, extracted by the current extracting part 524.

Also, the waveform operation part 525 makes the signs of the current waveform at the front location and the current waveform at the back location the same, by inverting the sign of the current waveform of the back location. Then, by adding up the current waveform of the back location with an inverted positivity/negativity, and the current waveform of the front location, the waveform operation part 525 generates a summation waveform of intra-axonal currents. By inverting the sign of the current waveform of the back location and then adding this to the current waveform of the front location, it is possible to prevent the current values from cancelling each other, and prevent a situation where a desired current waveform cannot be generated. Note that the current waveform at the back location and the current waveform at the front location may be displayed on the display device 40c.

The summation waveform of intra-axonal currents corresponds to the total amount of intra-axonal currents, and is equal to the total amount of inward currents that travel to a first virtual electrode of interest. That is, the summation waveform of intra-axonal currents represents a waveform corresponding to inward currents, and indicates the behavior of inward currents toward the depolarization site. The waveform display part 532 displays the summation waveform of intra-axonal currents generated by the waveform operation part 525, on the display device 40c, in association with each virtual electrode on the nerve axon.

Note that, for example, the back location of the first virtual electrode with electrode number 0 is outside the area where voxels are placed. Similarly, the front location of the first virtual electrode with electrode number 8 is located outside the area where voxels are placed. Therefore, the current extracting part 524 cannot properly extract the current component at the back location of the first virtual electrode of electrode number 0, and cannot properly extract the current component at the front location of the first virtual electrode of electrode number 8. In this case, the waveform operation part 525 does not generate a summation waveform of intra-axonal currents corresponding to the first virtual electrodes of electrode numbers 0 and 8. Therefore, no summation waveform of intra-axonal currents corresponding to the first virtual electrodes of electrode number 0 or 8 is generated.

FIG. 6 is an enlarged view that shows the current waveforms of inward currents and summation intra-axonal currents shown in FIG. 4. Although FIG. 4 and FIG. 6 show current waveforms that are obtained based on magnetic field measurement of a healthy person, for example, looking at the inward current waveforms of electrode numbers 3 and 4, the conduction velocity (27.4 m/s) of nerve action currents calculated from the peak latency is lower than the conduction velocity in the other inward current waveforms. Here, “peak latency” is the time it takes, after an electrical stimulation is applied to a peripheral nerve or the like, the current waveform to show a peak.

In contrast with this, looking at the waveforms of summation intra-axonal currents, the conduction velocity (74.4 m/s) between electrode numbers 3 and 4 is a neurophysiologically adequate value. Therefore, even if inward currents travel, for example, in a portion of the ulnar nerve and have to go through a narrow part between bones, such as the case with the cubital tunnel, and the inward currents' waveforms cannot be generated properly, it is still possible to provide an appropriate evaluation of nerve function based on current waveforms of summation intra-axonal currents, instead of current waveforms of inward currents.

FIG. 7 is a diagram that explains examples of various current waveforms in the ulnar nerve of a right arm when an electrical stimulation is applied to a subject's (patient's) right wrist. Parts that are the same as in FIG. 4 will not be described in detail here. FIG. 7 shows an example of an image displayed on the display device 40c of FIG. 1. In FIG. 7, too, the display device 40c displays a superimposed image including an X-ray image, current waveforms of intra-axonal currents at respective first virtual electrodes, current waveforms at respective second virtual electrodes (Rt), and current waveforms of summation intra-axonal currents.

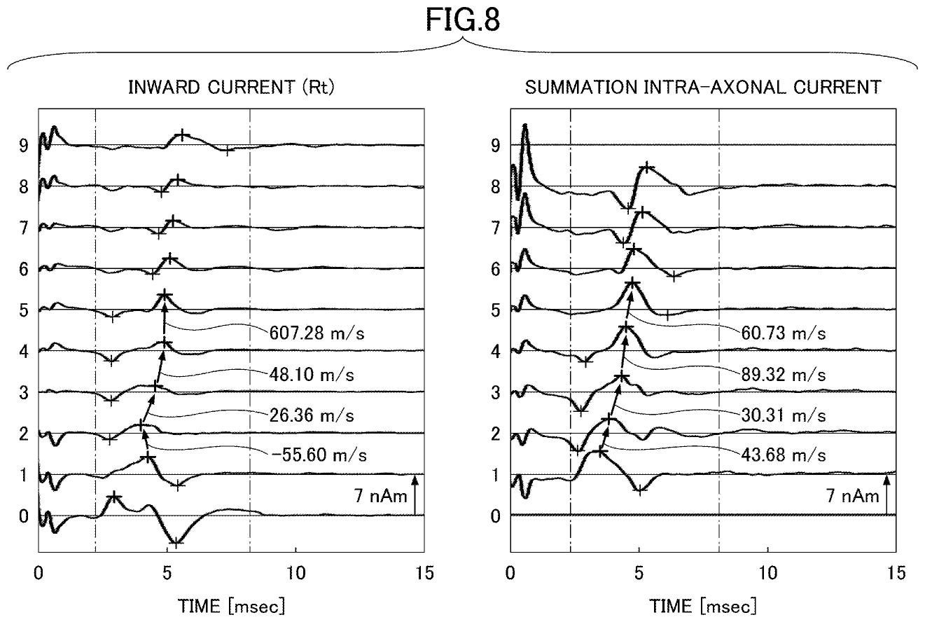

FIG. 8 is an enlarged view that shows the current waveforms of inward currents and summation intra-axonal currents shown in FIG. 7. Parts that are the same as in FIG. 6 will not be described in detail here. In some of the current waveforms of inward currents (Rt) shown in FIG. 8, the location of the peak is not clear; accordingly, the conduction velocity varies significantly. Therefore, it is difficult to identify the lesion site.

On the other hand, the peak location is clear in the current waveforms of summation intra-axonal currents, so that the conduction velocity can be calculated properly. Therefore, by observing the current waveforms of summation intra-axonal currents, the evaluator such as a doctor can provide an appropriate evaluation of nerve function and easily identify the lesion site. For example, the current waveforms of summation intra-axonal currents can identify the decrease in conduction velocity between electrode number 0 and electrode number 1 and between electrode number 2 and electrode number 3, which cannot be identified in the current waveforms of inward currents, so that the lesion site can be easily identified.

As described in the part of the summary of the invention, in some joints, inward currents cannot be calculated properly due to the influence of non-conductors such as bones. The arrangement of non-conductors in a joint changes depending on the flection angle of the joint. Therefore, the present inventors have conducted a clinical experiment to confirm the extent of the flection angle, at which the correct conduction velocity can be calculated based on current waveforms of summation intra-axonal currents.

The clinical experiment was conducted with five healthy people (subjects), by stimulating the ulnar nerve at the wrist joint and then measuring, at the elbow, the nerve magnetic field that is induced. The flection angle of the elbow upon the measurement was set to 30 degrees, 60 degrees, and 90 degrees. Here, the flection angle of the elbow indicates that the smaller the numerical value, the more straight the elbow is extended, and the larger the numerical value, the more bent the elbow is. Note that the normal conduction velocity at the elbow of the 5 subjects was confirmed in advance by the potential measurement method.

In the clinical experiment, the data processing device 50 of FIG. 1 was used to visualize the nerve action currents by applying spatial filtering to the measured magnetic field data. Then, using a first virtual electrode set provided at intervals of 20 mm along the conduction pathway of nerve action currents and second virtual electrodes provided to correspond with respective first virtual electrodes, intra-axonal current waveforms and inward current waveforms were calculated as in FIG. 4. Furthermore, the current waveforms of summation intra-axonal currents were calculated by the data processing device 50 by setting the first distance to 30 mm and the second distance to 30 mm in the same manner as in the method shown in FIG. 5.

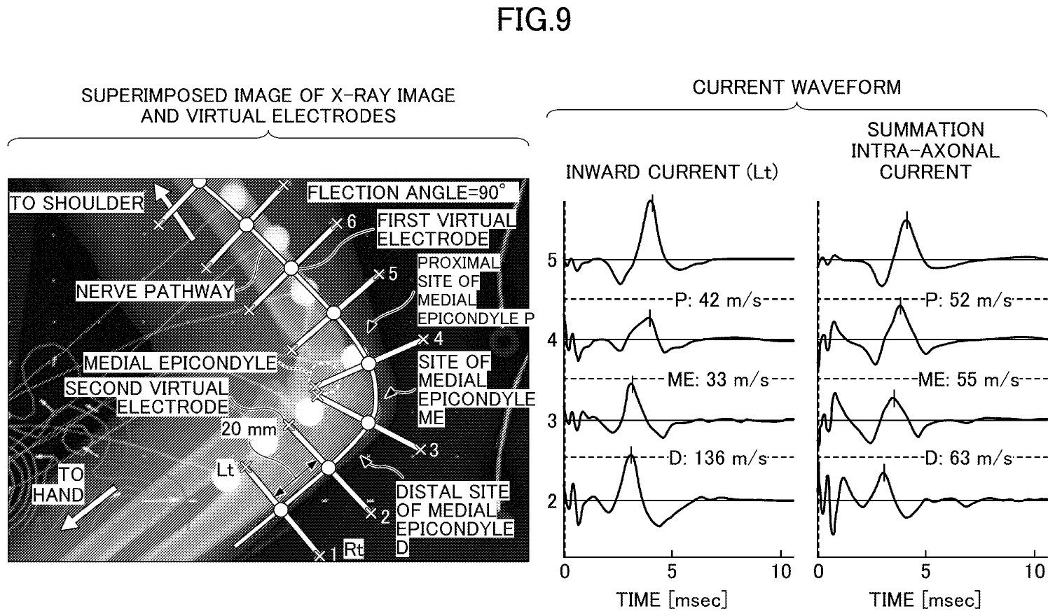

FIG. 9 is a diagram that illustrates examples of inward current waveforms and summation waveforms of intra-axonal currents at a left elbow when a stimulation is applied to the ulnar nerve at the left wrist joint of a healthy person. In the example shown in FIG. 9, the flection angle of the elbow is 90 degrees. Then, based on the peak latency of the inward current waveforms and summation waveforms of intra-axonal currents obtained, the conduction velocities of nerve action currents in the distal part of the medial epicondyle “D,” the site of the medial epicondyle “ME,” and the proximal site of the medial epicondyle “P” were compared. Here, the distal site of the medial epicondyle D is located between electrode numbers 2 and 3, the site of the medial epicondyle ME is located between electrode numbers 3 and 4, and the proximal site of the medial epicondyle P is located between electrode numbers 4 and 5.

In the inward current waveforms (conventional method), the respective conduction velocities of nerve action currents in the distal site of the medial epicondyle D, the site of the medial epicondyle ME and the proximal site of the medial epicondyle P were: P: 42 m/s; ME: 33 m/s; and D: 136 m/s. In the summation waveforms of intra-axonal currents (this method), these speeds were: P: 52 m/s; ME: 55 m/s; and D: 63 m/s. Note that the normal range of conduction velocity of nerve action currents is generally known to be between m/s and 80 m/s. Therefore, it was found that, although the inward currents (corresponding to the conventional method) in the example of FIG. 9 show abnormal values, the inward currents assume normal values when applying the technique of the present disclosure.

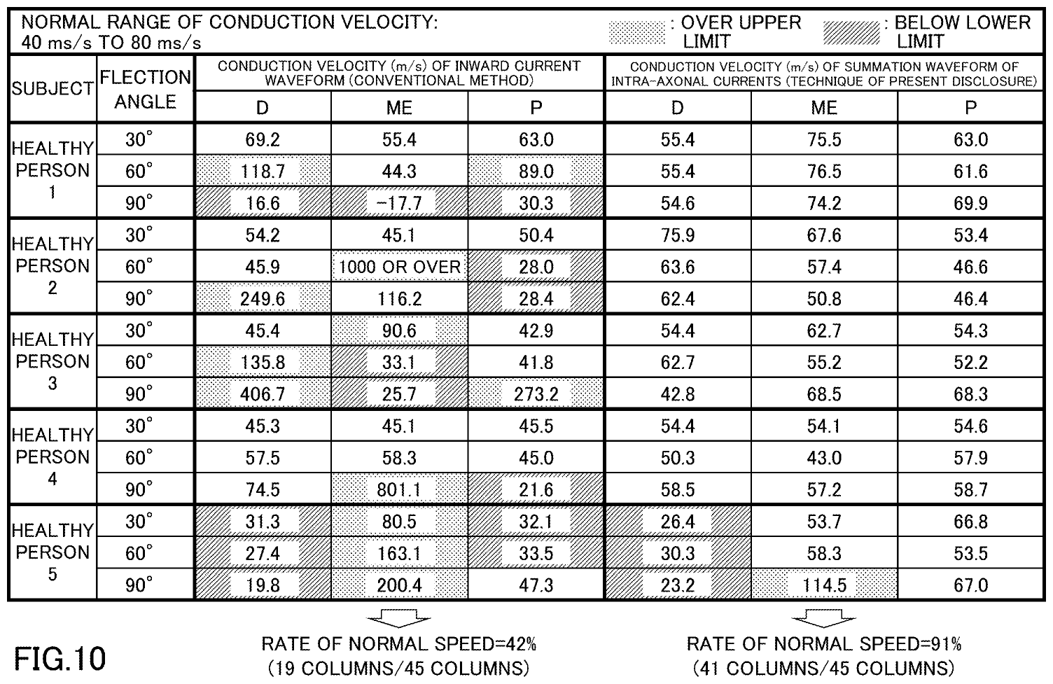

FIG. 10 is a diagram that illustrates the results of the clinical experiment with five healthy people. According to the conventional method of calculating conduction velocity based on the peak latency of inward current waveforms, normal values are shown at a rate of 42%. On the other hand, according to the method of the present disclosure, which calculates the conduction velocity based on the peak latency of summation intra-axonal currents, normal values are shown at a rate of 91%, and therefore it is likely that the nerve action at the elbows of the healthy subjects is reflected more accurately.

From the clinical experiment results shown in FIG. 9 and FIG. 10, it was confirmed that the summation waveforms of intra-axonal currents accurately reflect the subjects' nerve action at three flection angles (30 degrees, 60 degrees, and 90 degrees). Therefore, it is likely that, at any flection angle in the range of 30 degrees to 90 degrees, the summation waveform of intra-axonal current can accurately reflect the subjects' nerve action. That is, according to the method of the present disclosure, whereby the conduction velocity is calculated based on the peak latency of summation intra-axonal currents, the conduction velocity can be calculated accurately even if the arrangement of non-conductors in the elbow joint changes depending on the flection angle.

Furthermore, since the principle of the method of the present disclosure of calculating summation waveforms of intra-axonal currents does not rely upon the flection angle, even if the flection angle is less than 30 degrees or exceeds 90 degrees, it is still likely that the summation waveforms of intra-axonal currents can accurately reflect the subject's nerve action.

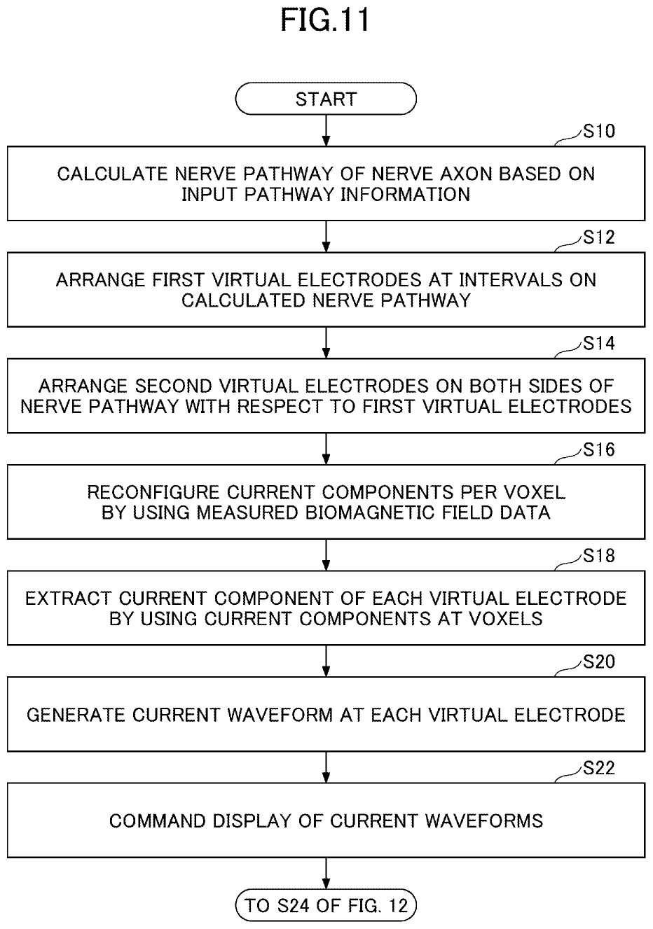

FIG. 11 to FIG. 13 are flowcharts that show example operations of the arithmetic part 520 in FIG. 1. The flows shown in FIG. 11 to FIG. 13 show example methods of controlling the data processing device 50 to function as a biomagnetic field measurement system, and show examples of processes by the control programs executed on the processor such as a CPU installed in the data processing device 50. Below, a case in which the body tissue to be evaluated is a nerve will be described.

First, in step S10, the pathway generating part 521 of the arithmetic part 520 calculates the nerve pathway of the nerve axon based on path information that is received as input at the location input part 511. For example, the location input part 511 receives, as path information, the coordinates of multiple locations on a morphological image displayed on the display device 40c, specified by the user viewing the image.

For example, the pathway generating part 521 generates a curve that runs along the nerve pathway from the coordinates of multiple locations specified by the user, and controls the image display part 531 to superimpose the generated curve over the morphological image and display the superimposed image. By this means, the nerve route can be set in accordance with the curved shape of the actual nerve axon.

Next, in step S12, the virtual electrode generating part 522 of the arithmetic part 520 arranges multiple first virtual electrodes, at intervals, on the path calculated by the pathway generating part 521 (that is, on the nerve pathway). Note that the number and spacing of the first virtual electrodes are entered in advance via the mouse 40a, the keyboard 40b, or the like. Next, in step S14, the virtual electrode generating part 522 of the arithmetic part 520 arranges second virtual electrodes on both sides of the nerve pathway with respect to the first virtual electrodes. As a result of this, first virtual electrodes and second virtual electrodes can be set at any locations along the nerve route set by the pathway generating part 521.

Next, in step S16, the reconfiguration analyzing part 523 of the arithmetic part 520 reconfigures the current components, on a per voxel basis, by using the biomagnetic field data of the subject P. Next, in step S18, the current extracting part 524 of the arithmetic part 520 extracts the current component at each virtual electrode by using the current components at the voxels, calculated by the reconfiguration analyzing part 523, based on the location relationship between each virtual electrode and voxel. For example, the current extracting part 524 extracts the current components at the first virtual electrodes along the nerve pathway as intra-axonal currents, and extracts the current components that travel from the second virtual electrodes to the first virtual electrodes on the nerve axon as inward currents.

Next, in step S20, the waveform operation part 525 of the arithmetic part 520 generates current waveforms that represent changes over time with the intra-axonal currents at the first virtual electrodes and the inward currents at the second virtual electrodes, extracted by the current extracting part 524. Next, in step S22, the waveform operation part 525 outputs a command to display the generated current waveforms on the display device 40c, to the waveform display part 532. As a result of this, as shown in FIG. 4 or FIG. 7, current waveforms of intra-axonal currents and inward currents are displayed on the display window in the screen of the display device 40c together with the superimposed image. Note that, at the time of step S22, the current waveforms of summation intra-axonal currents are not displayed.

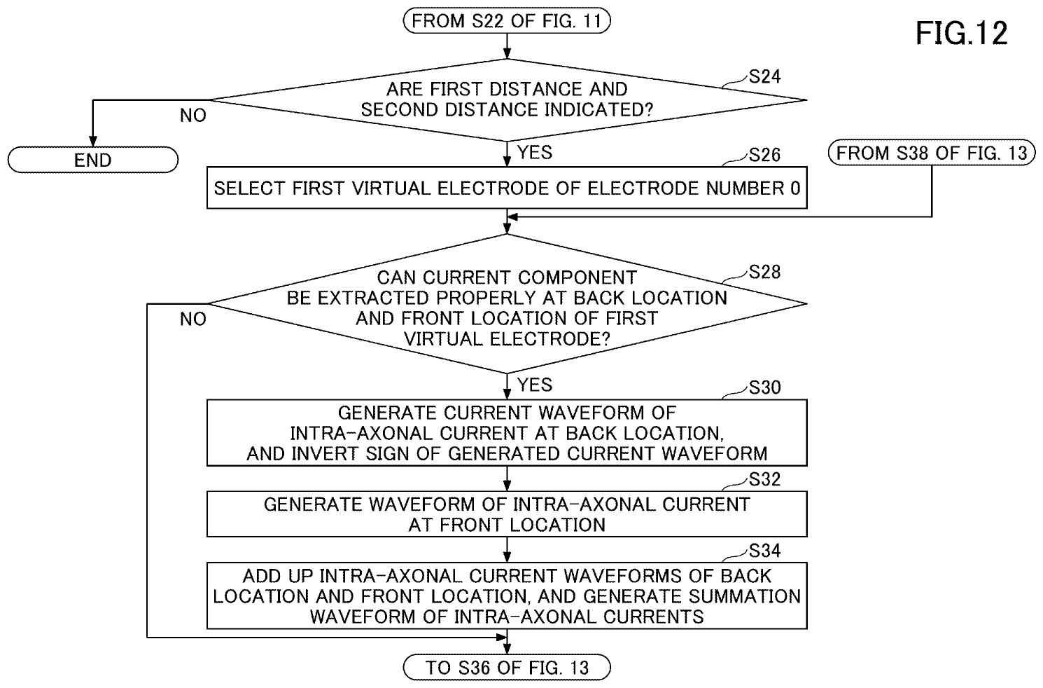

Next, in step S24 of FIG. 12, the waveform operation part 525 determines whether or not the first distance and the second distance are indicated. The waveform operation part 525 executes step S26 if the first distance and the second distance are indicated, and terminates the process shown in FIG. 11 to FIG. 13 if there are no indications of the first distance and the second distance. In this case, the current waveforms of summation intra-axonal currents in FIG. 4 or FIG. 7 are not displayed.

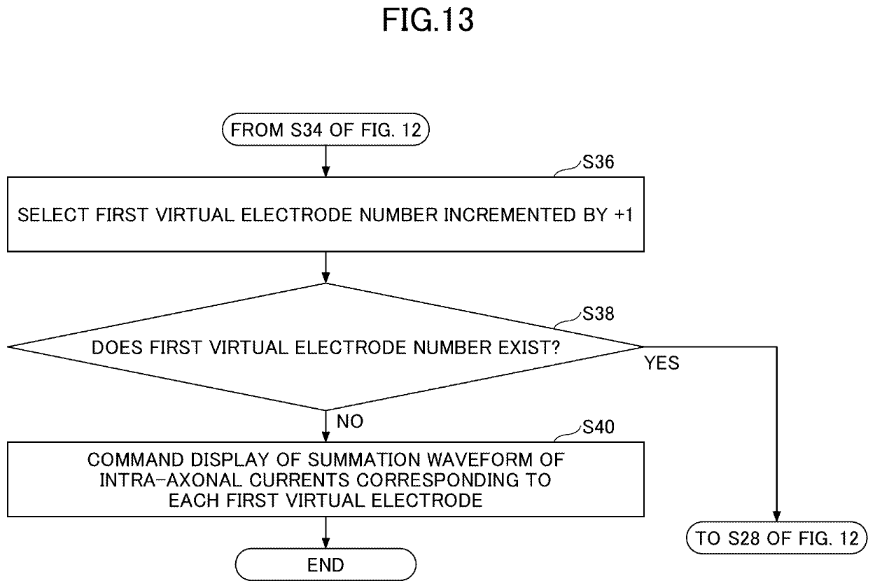

In step S26, the waveform operation part 525 selects the first virtual electrode of electrode number 0. Note that, in the process of FIG. 12 and FIG. 13, the first virtual electrodes are selected one by one, but the order of selection is by no means limited to the examples shown in FIG. 12 and FIG. 13.

Next, in step S28, the waveform operation part 525 determines whether the current components can be extracted appropriately at the back location and the front location that are the first and second distances apart from the first virtual electrode of the selected electrode number. If the current components can be extracted appropriately, the waveform operation part 525 executes step S30. If the current components cannot be extracted appropriately, the waveform operation part 525 executes step S36 of FIG. 13.

In step S30, the waveform operation part 525 extracts the current component at the back location of the first virtual electrode of the selected electrode number, and generates the current waveform of the intra-axonal current at the back location. Also, the waveform operation part 525 inverts the sign of the current waveform generated with respect to the intra-axonal current at the back location. Next, in step S32, the waveform operation part 525 extracts the current component at the front location of the first virtual electrode of the selected electrode number, and generates the current waveform of the intra-axonal current at the front location.

Next, in step S34, the waveform operation part 525 adds up the current waveform of the intra-axonal current at the back location with its sign inverted in step S30, and the current waveform of the intra-axonal current at the front location generated in step S30, thus generating a current waveform of a summation intra-axonal current.

Next, in step S36 of FIG. 13, the waveform operation part 525 selects the electrode number obtained by adding 1 to the currently selected electrode number. Next, in step S38, the waveform operation part 525 determines whether or not the electrode number selected in step S36 exists. The waveform operation part 525 executes step S28 of FIG. 12 if the electrode number exists, and executes step S40 if the electrode number does not exist.

In step S40, the waveform operation part 525 outputs a command to display the summation waveforms of intra-axonal currents corresponding to respective first virtual electrodes, generated in step S34, on the display device 40c, to the waveform display part 532. By this means, as shown in FIG. 4 or FIG. 7, summation waveforms of intra-axonal currents are displayed, in the display window in the screen of the display device 40c, together with the superimposed image, the intra-axonal current waveforms, and the inward current waveforms. After step S40, the process shown in FIG. 11 to FIG. 13 ends.

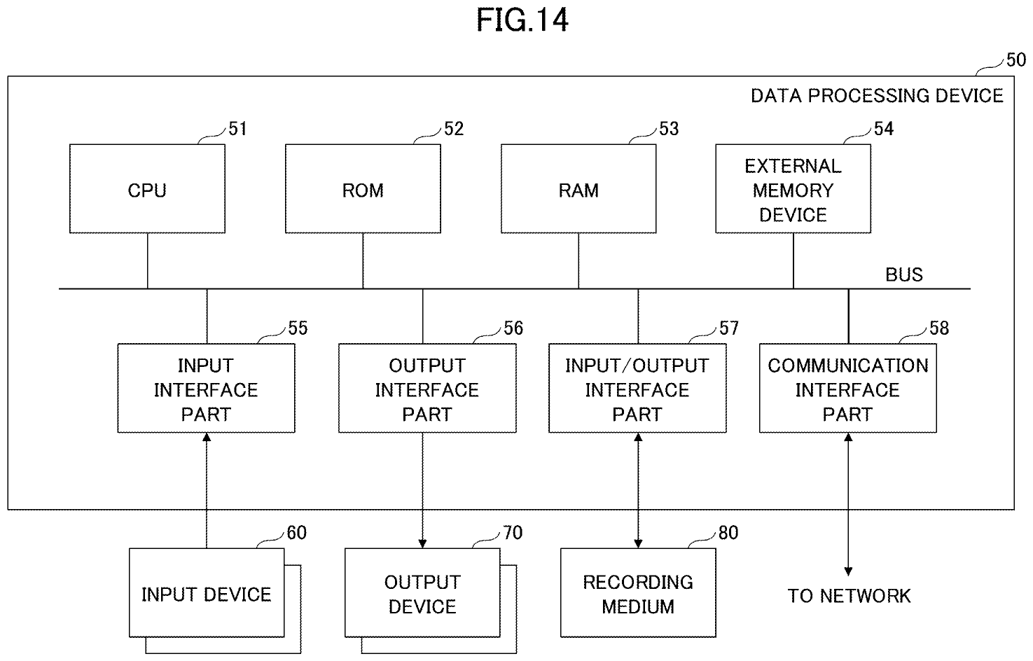

FIG. 14 is a block diagram that illustrates an example hardware structure of the data processing device 50 of FIG. 1. The data processing device 50 has a CPU 51, a ROM 52, a RAM 53, and an external memory device 54. Also, the data processing device 50 has an input interface part 55, an output interface part 56, an input/output interface part 57, and a communication interface part 58. For example, the CPU 51, the ROM 52, the RAM 53, the external memory device 54, the input interface part 55, the output interface part 56, the input/output interface part 57, and the communication interface part 58 are interconnected via a bus.

The CPU 51 executes various programs such as an OS (Operating System) and applications, and controls the overall operation of the data processing device 50. Also, the CPU 51 implements the method of controlling the data processing device 50 to function as a biomagnetic field measurement system by executing the above control programs. The CPU 51 is an example of a computer that executes control programs.

The ROM 52 holds various programs including control programs executed by the CPU 51, various parameters, and the like. The RAM 53 stores various programs executed by the CPU 51, data used in the programs, and so forth. The external memory device 54 is an HDD, an SSD, or the like, and stores various programs developed in the RAM 53.

The input interface part 55 is connected with an input device 60 that receives inputs from, for example, the user who operates the data processing device 50. For example, the input device 60 is the mouse 40a or the keyboard 40b of FIG. 1, a tablet, or the like. The output interface part 56 is connected with an output device 70 that outputs various images, text, graphics, and so forth that are generated by the data processing device 50. For example, the output device 70 is a display device 40c (FIG. 1) that displays display screens generated by various programs executed by the CPU 51, a printer, or the like.

A recording medium 80 such as a USB (Universal Serial Bus) memory is connected to the input/output interface part 57. For example, the recording medium 80 may store various programs such as control programs executed by the CPU 51. In this case, the programs are transferred from the recording medium 80 to the RAM 53 via the input/output interface part 57. Note that the recording medium 80 may be a CD-ROM, a DVD (Digital Versatile Disc: registered trademark), or the like. In this case, the input/output interface part 57 has an interface to support the recording medium 80 to be connected. The communication interface part 58 connects the data processing device 50 to a network or the like.

As described above, according to the present embodiment, even if inward currents travel, for example, in a portion of the ulnar nerve and have to go through a narrow part between bones, such as the case with the cubital tunnel, and the inward currents' waveforms cannot be generated properly, it is still possible to provide an appropriate evaluation of nerve function based on current waveforms of summation intra-axonal currents, instead of current waveforms of inward currents. In addition, even when the current waveforms of inward currents in body tissues such as skeletal muscles, cardiac muscles, smooth muscles, and so forth cannot be generated properly, it is still possible to evaluate the function of these body tissues appropriately by using the current waveforms of summation intra-cellular currents instead of inward current waveforms.

Multiple summation waveforms of intra-axonal currents are generated by adding up the current waveforms of current components, where these current components are located along a nerve pathway and are predetermined first and second set distances apart, on the nerve pathway, in front of and behind each of multiple first virtual electrodes. This makes it possible to detect changes in the conduction velocity of nerve action currents, and evaluate nerve function. By inverting the positivity/negativity of the current waveform at a back location and then adding this current waveform to the current waveform at a front location, it is possible to prevent the current values from cancelling each other, and prevent a situation where a desired current waveform cannot be generated.

The input control unit 510 receives as input the first distance (which corresponds to the back location) and the second distance (which corresponds to the front location) from the keyboard 40b or the like, so that the evaluator can freely set the locations to extract the intra-axonal currents that are used to generate summation waveforms of intra-axonal currents. That is, the evaluator can set the first distance and the second distance individually. This allows the evaluator to set a back location and a front location where appropriate summation waveforms of intra-axonal currents are obtained based on what he/she sees and learns.

Summation waveforms of intra-axonal currents are displayed on the display device 40c with at least one of inward current waveforms, intra-axonal current waveforms at first virtual electrodes, and a superimposed image, so that it is possible to evaluate nerve function with ease. In addition, in front of and behind a first virtual electrode, which is a location of interest located on the nerve pathway and designated on a superimposed image displayed on the display device 40c, virtual electrodes for generating summation waveforms of intra-axonal currents can be provided individually. This enables the evaluator to set the first distance and the second distance while checking the flection shape of the nerve pathway on the superimposed image and so forth, and set the virtual electrodes for generating summation waveforms of intra-axonal currents at appropriate locations.

The present disclosure has, for example, the following aspects:

<1> A biomagnetic field measurement system that has: circuitry; and a memory storing executable instructions which, when executed by the circuitry, cause the circuitry to: based on current components extracted from current signals calculated from a biomagnetic field signal, add up current waveforms of current components along a conduction pathway of an action current in a body tissue that is targeted for evaluation, and generate a current waveform for evaluating an intra-cellular current flowing in the conduction pathway, locations of the current components of the added current waveforms being predetermined set distances apart on the conduction pathway, in front of and behind a location of interest on the conduction pathway.

<2> The biomagnetic field measurement system according to above <1>, in which the executable instructions further cause the circuitry to: for each of a plurality of locations of interest on the conduction pathway, add up the current waveforms of the current components that are the predetermined set distances apart, on the conduction pathway, in front of and behind each of the locations of interest on the conduction pathway, and generate the current waveform for evaluating the intra-cellular current.

<3> The biomagnetic field measurement system according to above <1> or <2>, in which the executable instructions further cause the circuitry to: add up a current waveform at a location in front of the location of interest on the conduction pathway and a waveform at a location behind the location of interest on the conduction pathway, a sign of the waveform at the location behind the location of interest being inverted.

<4> The biomagnetic field measurement system according to one of above <1> to <3>, in which the executable instructions further cause the circuitry to: receive the predetermined set distances as input, the predetermined set distances being respective distances from the location of interest on the conduction pathway to the locations in front of and behind the location of interest on the conduction pathway, and being used to generate the current waveform for evaluating the intra-cellular current.

<5> The biomagnetic field measurement system according to above <4>, in which the executable instructions further cause the circuitry to: display the generated current waveform on a display device; at the location of interest on the conduction pathway, generate a current waveform of an inward current and a current waveform of the intra-cellular current, the inward current being a current component that travels from a vicinity of the conduction pathway toward the conduction pathway; and display the current waveform for evaluating the intra-cellular current on the display device, with at least one of the current waveform of the inward current and the current waveform of the intra-cellular current at the location of interest on the conduction pathway.

<6> The biomagnetic field measurement system according to above <5>, in which the executable instructions further cause the circuitry to: display a morphological image of a subject on the display device; receive as input the location of interest on the conduction pathway specified on the morphological image of the subject displayed on the display device; and set a virtual electrode at the location of interest on the conduction pathway received as input, and sets virtual electrodes for extracting the intra-cellular current that is used to generate the current waveform for evaluating the intra-cellular current, at locations that are the predetermined set distances apart, on the conduction pathway, in front of and behind the location of interest on the conduction pathway received as input.

<7> A method for controlling a biomagnetic field measurement system, including: based on current components extracted from current signals calculated from a biomagnetic field signal, adding up current waveforms of current components along a conduction pathway of an action current in a body tissue that is targeted for evaluation, and generating a current waveform for evaluating an intra-cellular current flowing in the conduction pathway, locations of the current components of the added current waveforms being predetermined set distances apart on the conduction pathway, in front of and behind a location of interest on the conduction pathway.

<8> A non-transitory computer-readable recording medium storing a program for controlling a biomagnetic field measurement system, the program, when executed by circuitry of a computer, causing the circuitry to: based on current components extracted from current signals calculated from a biomagnetic field signal, add up current waveforms of current components along a conduction pathway of an action current in a body tissue that is targeted for evaluation, and generate a current waveform for evaluating an intra-cellular current flowing in the conduction pathway, locations of the current components of the added current waveforms being predetermined set distances apart on the conduction pathway, in front of and behind a location of interest on the conduction pathway.

Although the present disclosure has been described above based on an embodiment, the present disclosure is by no means limited to the requirements shown in the above embodiment. These points can be changed within the scope of the present disclosure, and can be determined properly according to the mode of implementation.

What is claimed is:

1. A biomagnetic field measurement system comprising:
circuitry; and
a memory storing executable instructions which, when executed by the circuitry, cause the circuitry to:
based on current components extracted from current signals calculated from a biomagnetic field signal, add up current waveforms of current components along a conduction pathway of an action current in a body tissue that is targeted for evaluation, and generate a current waveform for evaluating an intra-cellular current flowing in the conduction pathway, locations of the current components of the added current waveforms being predetermined set distances apart on the conduction pathway, in front of and behind a location of interest on the conduction pathway.

2. The biomagnetic field measurement system according to claim 1, wherein the executable instructions further cause the circuitry to:
for each of a plurality of locations of interest on the conduction pathway, add up the current waveforms of the current components that are the predetermined set distances apart, on the conduction pathway, in front of and behind each of the locations of interest on the conduction pathway, and generate the current waveform for evaluating the intra-cellular current.

3. The biomagnetic field measurement system according to claim 1, wherein the executable instructions further cause the circuitry to:
add up a current waveform at a location in front of the location of interest on the conduction pathway and a waveform at a location behind the location of interest on the conduction pathway, a sign of the waveform at the location behind the location of interest being inverted.

4. The biomagnetic field measurement system according to claim 1, wherein the executable instructions further cause the circuitry to:
receive the predetermined set distances as input, the predetermined set distances being respective distances from the location of interest on the conduction pathway to the locations in front of and behind the location of interest on the conduction pathway, and being used to generate the current waveform for evaluating the intra-cellular current.

5. The biomagnetic field measurement system according to claim 4, wherein the executable instructions further cause the circuitry to:
display the generated current waveform on a display device;
at the location of interest on the conduction pathway, generate a current waveform of an inward current and a current waveform of the intra-cellular current, the inward current being a current component that travels from a vicinity of the conduction pathway toward the conduction pathway; and
display the current waveform for evaluating the intra-cellular current on the display device, with at least one of the current waveform of the inward current and the current waveform of the intra-cellular current at the location of interest on the conduction pathway.

6. The biomagnetic field measurement system according to claim 5, wherein the executable instructions further cause the circuitry to:
display a morphological image of a subject on the display device;
receive as input the location of interest on the conduction pathway specified on the morphological image of the subject displayed on the display device; and
set a virtual electrode at the location of interest on the conduction pathway received as input, and sets virtual electrodes for extracting the intra-cellular current that is used to generate the current waveform for evaluating the intra-cellular current, at locations that are the predetermined set distances apart, on the conduction pathway, in front of and behind the location of interest on the conduction pathway received as input.

7. A method for controlling a biomagnetic field measurement system, comprising:
based on current components extracted from current signals calculated from a biomagnetic field signal, adding up current waveforms of current components along a conduction pathway of an action current in a body tissue that is targeted for evaluation, and generating a current waveform for evaluating an intra-cellular current flowing in the conduction pathway, locations of the current components of the added current waveforms being predetermined set distances apart on the conduction pathway, in front of and behind a location of interest on the conduction pathway.

8. A non-transitory computer-readable recording medium storing a program for controlling a biomagnetic field measurement system, the program, when executed by circuitry of a computer, causing the circuitry to:
based on current components extracted from current signals calculated from a biomagnetic field signal, add up current waveforms of current components along a conduction pathway of an action current in a body tissue that is targeted for evaluation, and generate a current waveform for evaluating an intra-cellular current flowing in the conduction pathway, locations of the current components of the added current waveforms being predetermined set distances apart on the conduction pathway, in front of and behind a location of interest on the conduction pathway.
